(19) United States
(12) Patent Application Publication
(10) Pub. No.: US 2021/0348211 A1
(43) Pub. Date: Nov. 11, 2021

(54) METHOD FOR DEFINING STAGES OF DEVELOPMENT OF CYANOBACTERIAL BLOOM

(71) Applicant: NANJING INSTITUTE OF GEOGRAPHY & LIMNOLOGY. CHINESE ACADEMY OF SCIENCES, Nanjing, Jiangsu Province (CN)

(72) Inventors: Xiaoli SHI, Nanjing, Jiangsu Province (CN); Kaining CHEN, Nanjing, Jiangsu Province (CN); Yang YU, Nanjing, Jiangsu Province (CN); Zhen YANG, Nanjing, Jiangsu Province (CN); Min ZHANG, Nanjing, Jiangsu Province (CN)

(73) Assignee: NANJING INSTITUTE OF GEOGRAPHY & LIMNOLOGY. CHINESE ACADEMY OF SCIENCES, Nanjing, Jiangsu Province (CN)

(21) Appl. No.: 17/049,590

(22) PCT Filed: Apr. 16, 2019
(86) PCT No.: PCT/CN2019/082891

§ 371 Date: Oct. 22, 2020

(30) Foreign Application Priority Data
Apr. 28, 2018 (CN) 201810298866.0
Apr. 28, 2018 (CN) 201810399140.9
Apr. 28, 2018 (CN) 201810400286.2
Apr. 28, 2018 (CN) 201810400311.5

(51) Int. Cl. C12Q 1/686; G01N 33/00

(52) U.S. Cl. CPC C12Q 1/686; G01N 33/0098

(57) ABSTRACT

Disclosed is a method for defining the stages of development of a cyanobacterial bloom, wherein the development of a cyanobacterial bloom is divided into five stages: a cyanobacteria wintering period, a resuscitation period, a rapid growth period, an outbreak period and a cyanobacteria decline period; the wintering period is defined based on the concentration/content ratio of algocyan/chlorophyll a; the resuscitation period is defined based on the relative expression levels of the phycocyanin synthesis gene PC-IGS, the algal toxin synthesis gene mcyA and the gas vesicle synthesis gene gvpC in the surface sediment; the cyanobacteria rapid growth period is defined based on the relative expression level of the ftsZ gene; the cyanobacteria outbreak period is defined based on the wind speed; and the cyanobacteria decline period is defined based on the relative expression level of the nblA gene and the ratio of glucose to neutral polysaccharides in dissolved organic matter.

### TECHNICAL FIELD

The present invention relates to the technical field of water environment treatment and cyanobacterial bloom control, particularly to a method for defining the stages of development of a cyanobacterial bloom.

### BACKGROUND ART

Lake eutrophication and cyanobacterial bloom is a major environmental problem. Currently, cyanobacterial blooms in large shallow lakes are controlled mainly in summer when the cyanobacterial biomass is large, but the control of cyanobacterial blooms should address the root cause. It is necessary to kill and control cyanobacteria during the outbreak of a cyanobacterial bloom and also take measures to inhibit their development when cyanobacteria are dead or in the early stage of growth. Therefore, it is necessary to define the various stages of development of a cyanobacterial bloom and take different measures in each development stage of the cyanobacterial bloom according to the growth law of cyanobacteria.

At present, the four-stage theory of cyanobacterial bloom development: sinking-dormancy-resuscitation-floating is popular. December to February is defined as a sinking and dormancy period and the dominant factors are low temperature and darkness. March to April is defined as a floating and resuscitation period and the dominant factors are temperature, oxygen and waves. April to September is defined as a mass growth period and the dominant factor is matter energy. April to November is a floating and accumulation period and the dominant factor is hydrometeorology. However, the current four-stage theory was proposed based on a research model, which is a hypothesis of the cause of cyanobacterial bloom, and the definition of each growth period is still relatively vague.

### SUMMARY OF THE INVENTION

The objective of the present invention is to provide a method for defining the stages of development of a cyanobacterial bloom, which divides the development of a cyanobacterial bloom into five stages and provides a method for discriminating the stages based on key factors and thresholds.

In order to achieve the above technical objective, the present invention adopts the following technical solution:

A method for defining the stages of development of a cyanobacterial bloom, comprising: dividing the development of a cyanobacterial bloom into five stages: a cyanobacteria wintering period, a resuscitation period, a rapid growth period, an outbreak period and a cyanobacteria decline period; and the definition steps are as follows:

Step (1): Collect mixed water samples of water columns from the upper, middle and lower layers of the water body, overlying water samples and sediment samples at lake sampling points; determine the concentrations/contents of algocyan and chlorophyll a in the water columns, the overlying water and the sediment; calculate the concentration/content ratios of algocyan/chlorophyll a in the water columns, the overlying water and the sediment respectively; and define the time as a cyanobacteria wintering period when the concentration/content ratios of algocyan/chlorophyll a in the sediment, the overlying water and the water columns at each sampling point are all less than 1;

Step (2): Starting from the cyanobacteria wintering period, collect surface sediment samples from the lake sampling points, extract RNA from the surface sediment samples and determine the relative expression levels of phycocyanin synthesis gene PC-IGS, algal toxin synthesis gene mcyA and gas vesicle synthesis gene gvpC; and define that the cyanobacteria enter a resuscitation period when the relative expression levels of the genes satisfy relative expression level of PC-IGS>3, relative expression level of mcyA>0.03 and relative expression level of gvpC>0.004 at the same time;

Step (3): Starting from the cyanobacterial resuscitation period, collect complete water column algae samples from the lake sampling points, extract RNA and determine the relative expression level of the ftsZ gene; culture indoor the microcystis isolated from the same water column algae samples, establish a regression equation of microcystis growth rate μ and the relative expression level of the ftsZ gene, determine a rapid growth period of microcystis, obtain the relative expression level of the ftsZ gene at the moment as a threshold of the cyanobacteria rapid growth period window, and define that the cyanobacteria enter a lake cyanobacteria rapid growth period if the relative expression level of the ftsZ gene is greater than this threshold;

Step (4): Starting from the cyanobacteria rapid growth period, monitor the wind speed of each monitoring point in the lake, use 3.1 m/s as a threshold for defining an outbreak period and define the time as a cyanobacteria decline period when the wind speed is less than 3.1 m/s;

Step (5): Starting from the cyanobacteria rapid growth period, collect complete water column samples from the lake sampling points, determine the relative expression level of the nblA gene and the ratio of glucose to neutral polysaccharides in dissolved organic matter, and define that the cyanobacteria enter a decline period when the relative expression level of the nblA gene reaches 2.4 and the ratio of dissolved glucose to neutral polysaccharides in the water body is greater than 30%.

Further, the step (1) further comprises: determining the sampling points in the lake as lake cyanobacteria wintering areas if the content ratio of algocyan/chlorophyll a in the sediment, the concentration ratio of algocyan/chlorophyll a in the overlying water and the concentration ratio of algocyan/chlorophyll a in the water columns at these points are all less than 1, and estimating and demarcating the area ranges based on the data of the monitoring points.

At the step (1), the earliest day when the concentration/content ratios of algocyan/chlorophyll a in the sediment, the overlying water and the water columns at each sampling point are all less than 1 is the first day of the cyanobacteria wintering period. The 10 days before and after the first day of the cyanobacteria wintering period is a window period for taking control measures. According to the defined window period and the foregoing estimated and demarcated wintering area ranges, cyanobacteria can be treated pertinently in the defined window period and the demarcated area ranges.

Further, at the step (1), the water sample from the upper layer of the water body refers to the water from the surface layer of the water body to 20 cm underwater; the water sample from the lower layer of the water body refers to the water 20 cm from the bottom layer; the middle section refers to a water sample from the middle layer of the water body; the overlying water refers to the water in the surface layer of sediment.

Further, the step (2) further comprises sequencing according to the relative expression levels of the three genes at different sampling points of the lake, partitioning the lake through spatial grid division and spatial interpolation, and determining the areas where the relative expression level of PC-IGS>3, the relative expression level of mcyA>0.03 and the relative expression level of gvpC>0.004 as resuscitation areas of wintering cyanobacteria. It is preferred to use GIS to layout grids, use ArcGIS software for spatial interpolation and data storage and use the inverse distance weight interpolation method for spatial interpolation.

At the step (2), starting from the cyanobacteria wintering period, the earliest day when the relative expression level of PC-IGS>3, the relative expression level of mcyA>0.03 and the relative expression level of gvpC>0.004 in surface sediment is the first day of the resuscitation period. The 15 days before and after the first day of the resuscitation period is a window period for taking control measures. According to the defined window period and the foregoing estimated and demarcated resuscitation area ranges, cyanobacteria can be treated pertinently in the defined window period and the demarcated area ranges.

Further, at the step (3), the isolated microcystis population taken from the lake is divided into several treatment groups and cultured in different growth environments and samples are taken every day to measure the density changes of the algae cells to obtain growth rates of the algae cells in different growth environments and in different growth cycles; the calculation formula of the growth rate is: ρ=(InNₜ−InN₀)/t, where Nₜ and N₀ represent the cell density at time t and the cell density at the initial time respectively; and the culture cycle is 30 days and microcystis in each treatment group is sampled to obtain the relative expression level of the ftsZ gene in unit cells.

Further, at the step (5), neutral polysaccharides include ribose, xylose, arabinose, rhamnose, fucose, galactose, glucose and mannose.

Further, the present invention adopts the fluorescent quantitative PCR method to determine the relative expression levels of genes.

The present invention provides a method for defining the stages of development of a cyanobacterial bloom based on the growth law of cyanobacterial bloom throughout the year, discriminates the key parameters and thresholds of cyanobacterial bloom in different stages of the whole process through field annual investigation and monitoring and determines the window periods for implementing algae control and removal technologies in various stages. The method based on the present invention can realize the definition of the development stages of a cyanobacterial bloom, thereby realizing targeted algae removal and control in each stage and finally realizing whole-process algae control.

### BRIEF DESCRIPTION OF THE DRAWINGS

FIG. 1 shows the annual change in the content ratio of algocyan/chlorophyll in the bottom sediment of different lake areas in the Taihu Lake;

FIG. 2 shows the annual change in the content ratio of algocyan/chlorophyll in the overlying water of different lake areas of the Taihu Lake;

FIG. 3 shows the annual change in the concentration ratio of algocyan/chlorophyll in the water bodies of different lake areas of the Taihu Lake;

FIG. 4 shows the layout method of sampling points in the Taihu Lake;

FIG. 5 shows the relative expression level of the PC-IGS gene in the surface sediment of the Taihu Lake;

FIG. 6 shows the annual change in the relative expression level of the gvpC gene in the surface sediment of the Taihu Lake;

FIG. 7 shows the annual change in the relative expression level of the mcyA gene in the surface sediment of the Taihu Lake;

FIG. 8 shows the relation between the expression level of the ftsz gene and the growth rate of microcystis in the water columns of the sampling points of the Taihu Lake;

FIG. 9 shows the in situ growth rate of cyanobacteria in the Taihu Lake;

FIG. 10 shows the annual change law of the relative expression level of the phycobilisome degradation protein gene in the Taihu Lake;

FIG. 11 shows the annual change diagram of the ratio of glucose to neutral polysaccharides in the dissolved organic matter of the Taihu Lake.

### DETAILED DESCRIPTION

The embodiments take the Taihu Lake as an example to specifically illustrate the method of the present invention for defining the stages of development of a cyanobacterial bloom.

Step (1): Mixed water samples of water columns from the upper, middle and lower layers of the water body, overlying water samples and sediment samples at the sampling points of the Taihu Lake are collected;

Samples are collected at the sampling points from the end of autumn to the beginning of spring, generally from November to March each year according to the meteorological conditions.

The sampling frequency is once every half a month. Mixed samples from the upper, middle, and lower layers of the water body, samples from the overlying water and samples from the sediment surface layer at a depth of 15 cm are collected. The sediment samples are collected with a cylindrical sampler in a pipe diameter of greater than 15 cm.

The collected samples are brought back to the laboratory to analyze and determine the content/concentration of chlorophyll a and algocyan in the sediment, the overlying water and the water columns and calculate the concentration/content ratios of algocyan/chlorophyll a in the water columns, the overlying water and the sediment respectively.

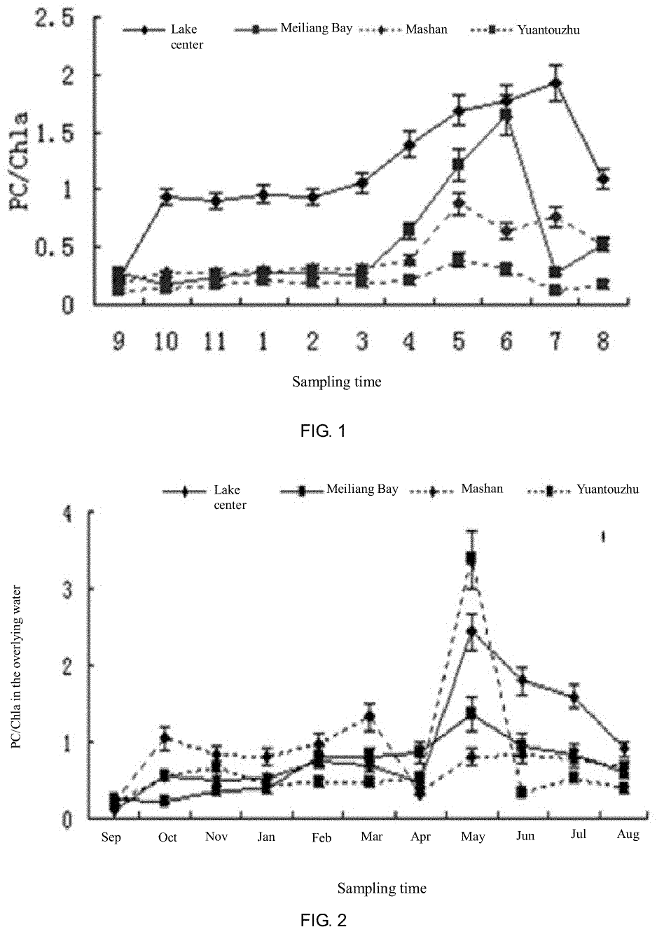

As shown in FIG. 1, the content ratio of algocyan/chlorophyll in the sediment (PC/Chla) represents the proportion of cyanobacteria to total algae in the sediment. The annual investigation results of sediment PC/Chla in different lake areas of the Taihu Lake show that from September to next March, the ratio is relatively stable, less than 1 in all lake areas, and in April, with the increase of temperature, the activity of cyanobacteria in the sediment rises and cyanobacteria begin to grow in large quantities and the sediment PC/Chla begin to rise rapidly.

FIG. 2 shows the annual change in the concentration ratio of algocyan/chlorophyll in the overlying water of different lake areas (sampling points) of the Taihu Lake.

In the wintering period, the cyanobacteria in the water columns are mostly distributed in the lower layer of the water body. The annual change law in the concentration ratio of algocyan/chlorophyll in the overlying water shows that from September to next April, this ratio is about less than 1 in all lake areas, and in May, due to the rise of temperature, the cyanobacteria in the overlying water begin to grow rapidly and the cyanobacteria in the sediment also begin to be resuscitated, resulting in a rapid increase in the concentration ratio of algocyan/chlorophyll in the overlying water to a peak.

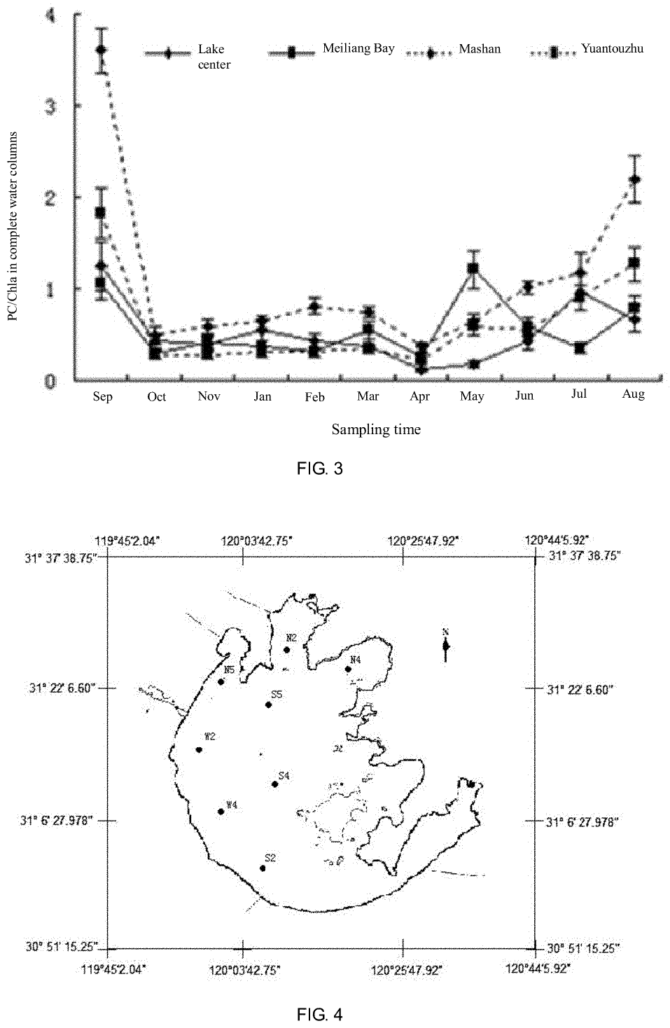

FIG. 3 shows the annual change in the concentration ratio of algocyan/chlorophyll in the water bodies of different lake areas (sampling points) of the Taihu Lake.

The planktonic algae communities in the water body of the Taihu Lake will undergo succession each year, diatoms are dominant in winter and green algae are dominant in spring, so the concentration ratio of algocyan/chlorophyll in the entire water columns is relatively low from October to next April, less than 1 in all the investigated lake areas. Compared with other types of algae, cyanobacteria are more suitable to grow in water with higher temperature. In May, cyanobacteria enter a rapid growth period. The concentration ratio of algocyan/chlorophyll in the water columns increases rapidly.

Based on the foregoing three indicators, it is defined that the cyanobacteria wintering period starts from September; in order to effectively eliminate wintering cyanobacteria, the 10 days before and after the first day of the wintering period are determined as a window period for taking control measures.

The sampling points in the lake are defined as lake cyanobacteria wintering areas if the content ratio of algocyan/chlorophyll a in the sediment, the concentration ratio of algocyan/chlorophyll a in the overlying water and the concentration ratio of algocyan/chlorophyll a in the water columns at these points are all less than 1, and the area ranges are estimated and demarcated based on the data of the monitoring points

Step (2): 8 sampling points as shown in FIG. 4 are distributed throughout the Taihu Lake. The sampling frequency is to collect bottom sediment samples once a month. The collected bottom sediment samples are quickly mixed uniformly, added with an RNA later protection solution, quickly put into liquid nitrogen for storage, brought back to the laboratory and stored at −70° C. until RNA is extracted.

The RNA in the bottom sediment is extracted with FastRNA Pro Soil Direct Kit by the following operation steps:

Weigh 0.5-1 g of the sediment, add it into a 2 mL purple lysis tube E (Lysing Matrix E, containing glass beads with diameters of 1.2 mm, 0.074 mm and 4 mm), then add 1 mL of soil lysis buffer to the lysis tube E and invert the tube several times so that the sediment and the glass beads are suspended in the lysis buffer. Place the lysis tube E containing the sediment and lysis buffer in a Fast Prep-24 sample treatment instrument, set the parameters of the Fast Prep-24 sample treatment instrument, 6 m/s, 40 s, to break the sediment. Then centrifuge at room temperature and 14,000×g for 5 min. Transfer the obtained supernatant to a new centrifuge tube, and then add 750 μL of phenol:chloroform (1:1) to the new centrifuge tube and vortex for 10 s. Keep the tube in a warm bath at room temperature for 5 min to decompose nuclear protein and increase RNA purity. Then centrifuge at 14,000×g and 4° C. for 5 min. After centrifugation, carefully transfer the supernatant to a new centrifuge tube without destroying the intermediate material. Add 200 μL of RNA inhibitor removing solution to a new centrifuge tube, invert the tube 5 times to mix the solution well and then centrifuge the solution at 14000×g and room temperature for 5 min. Transfer the supernatant to a new centrifuge tube, and add 660 μL of frozen 100% isopropanol to the sample, invert the tube 5 times until thorough mixing, and store it at −20° C. overnight. Centrifuge at 4° C. and 14000 g for 15 min, and discard the supernatant. Wash the precipitate with 500 μL of frozen 70% ethanol, pour off the 70% ethanol, and dry the precipitate at room temperature in the air for 5 min. Suspend the obtained RNA in 200 μL of DEPC water. The RNA obtained at the above steps should be cleaned. Add 600 μL of RNAMATRIX binding solution and 10 μL of RNAMATRIX suspension to the DEPC water containing RNA. After even mixing, keep it in a warm bath at room temperature for 5 min, centrifuge to precipitate RNA bound with RNAMATRIX and pour off the supernatant. Add 500 μL of RNAMATRIX washing solution to the RNA precipitate bound with RNAMATRIX, continue to centrifuge to remove the supernatant, centrifuge again to remove the remaining washing solution and then dry at room temperature for 5 min. Add 50 μL of DEPC water and vortex to suspend RNAMATRIX, and after centrifugation, add the RNA-containing supernatant to a new RNase-free centrifuge tube.

After extraction of RNA from the bottom sediment, conduct reverse transcription using Transciptor First Strand cDNA Synthesis Kit. Add 1 μL of Anchored-oligo(dT), 2 μL of random hexamer primers, 400 ng of RNA and an appropriate amount of PCR-grade water to about 400 ng of RNA sample to make the total volume be 13 μL. After mixing, keep the mixture in a 65° C. warm bath of a PCR machine for 10 min. After the warm bathing, add 4 μL of transcriptor reverse transcriptase reaction buffer, 0.5 μL of RNA inhibitor, 2 μL of deoxynucleotide mix and 0.5 μL of transcriptor reverse transcriptase in turn, mix well and put the mixture on a PCR amplifier for reaction. Set the program of the PCR amplifier as 25° C. 10 min, 55° C. 30 min and 85° C. 5 min. Place the PCR tube on ice immediately after the reaction and store the obtained cDNA at −20° C. for later use.

Place the PCR tube on ice immediately after the reaction and store the obtained cDNA at −20° C. for later use. The primers designed for each target gene are:

PC-IGS gene
188F 5′-GCTACTTCGACCGCGCC-3′;
254R 5′-TCCTACGGTTTAATTGAGACTAGCC-3′;
mcyA gene
mcyA-F 5′-AAAATTAAAAGCCGTATCAAA-3′;
mcyA-R 5′-AAAAGTGTTTTATTAGCGGCTCAT-3′);
gypC gene
gypC-F 5′-TGCTTTGCGTCAGTCTTTCC-3′;
gypC-R 5′-TCCTTCACCTGTTTGGCTCT-3′);

The cDNA template with 16S rRNA as a primer is diluted 10 times, while other cDNA templates are not diluted.

The amplification procedure is as follows: The qRT-RCR reaction system is 25 μL, which contains 12.5 μL of 2×QuantiFast SYBR Green PCR Master Mix, 2 μM of primers, 1 μL of template and 10.5 μL of RNase-free water. The PCR reaction is performed in replex⁴ (Eppendorf) fluorescent quantitative PCR amplifier. The amplification procedure is as follows: pre-denature at 95° C. for 3 min, denature at 95° C. for 15 s, anneal at 60° C. for 30 s and extend at 72° C. for 45 s, in 40 cycles. After PCR is completed, the corresponding threshold cycle value (“Ct value” for short, defined as the number of cycles experienced when the fluorescence signal reaches the set threshold. The copy number of the target gene is negatively correlated with the Ct value) is derived. The gene expression level is evaluated with the Ct value of the reaction. 16S rRNA is used as a positive internal control gene in the PCR process to correct the cell copy number of the PCR template, thereby eliminating the amount of sample addition between groups.

Fluorescence quantitative PCR amplification: A fluorescence quantitative PCR amplification curve consists of three stages: fluorescent background signal stage, fluorescent signal exponential amplification stage and plateau stage. After the fluorescence quantitative PCR, the Ct value can be derived directly.

The relative expression level of the gene is expressed with 2^{-ΔΔCt}. The calculation formula of ΔΔCt is as follows: ΔΔCt=(Ct_{target gene}-Ct_{16S rRNA})_{field}−(Ct_{target gene}−Ct_{16S rRNA}) control.

After the relative expression levels of the genes are determined, these three indicators are comprehensively analyzed to determine the resuscitation window period of wintering cyanobacteria. The specific method is as follows: Draw a curve with time as the x-axis and the relative expression levels of the genes as the y-axis. According to the curves, changes in the expression parameters of the three genes that are directly related to the activity recovery and floating of cyanobacteria are comprehensively analyzed. When the relative expression level of the phycocyanin transcribed spacer gene PC-IGS>3, the expression level of the algal toxin gene (mcyA)>0.03 and the expression level of the gas vesicle gene (gvpC)>0.004 in the cyanobacteria of the bottom sediment water body, it is comprehensively analyzed and determined that the wintering cyanobacteria in the bottom sediment enter a resuscitation period. The upper threshold (the earliest day that meets PC-IGS>3, mcyA>0.03 and gvpC>0.004 at the same time) of the resuscitation period minus 15 days is determined as a window period for taking measures to eliminate germplasm sources.

The expressions of the phycocyanin gene (PC-IGS), the algal toxin gene (mcyA) and the gas vesicle gene (gvpC) all start in winter and reach a maximum in spring, but the expressions of these three genes also differ in time. The expression of the gas vesicle gene is earlier than the expression of phycocyanin gene because with the increase in the expression level of the gas vesicle gene, cyanobacteria quickly synthesize gas vesicles, thereby driving the cyanobacteria to float from the bottom sediment up to the surface of the water body and subsequently enabling the cyanobacteria to acquire suitable growth conditions and nutritive salts. Only by then does the phycocyanin gene begin to be expressed rapidly.

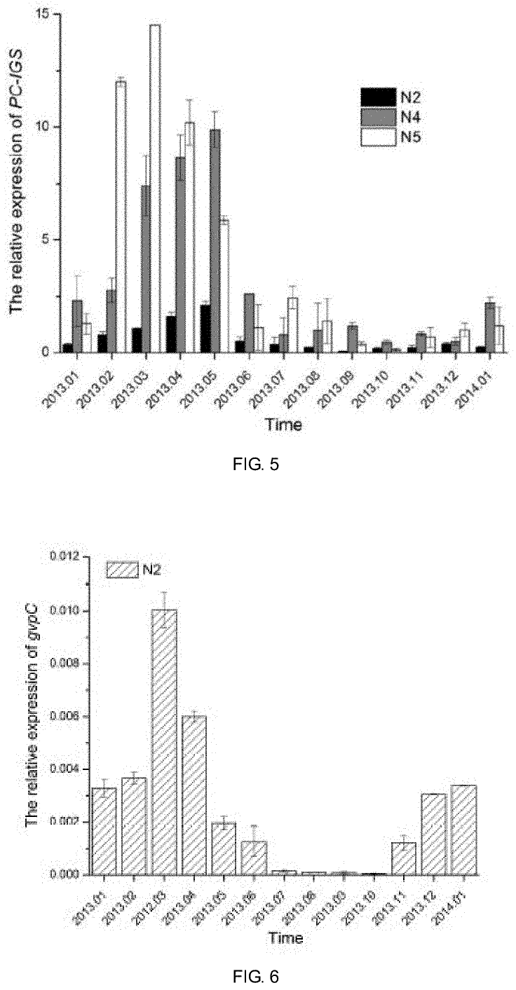

FIG. 5 shows the relative expression level of the PC-IGS gene in the surface sediment of the Lake Taihu. The relative expression level of the phycocyanin transcribed spacer gene (PC-IGS) gradually increases from January to May and quickly reaches its maximum value in May, which is throughout the entire wintering resuscitation period. After the formation of a bloom, the expression of the phycocyanin gene gradually declines, which indicates that when microcystis enters a bloom period, it can float to the lake surface to obtain maximum illumination without continuing to synthesize more phycocyanin.

FIG. 6 shows the annual change in the relative expression level of the gvpC gene in the surface sediment of the Taihu Lake. In the wintering resuscitation stage of cyanobacteria, the gas vesicle gene transcription level gradually increases and reaches its maximum value in March, but in the biomass increase and floating stage of cyanobacteria, the expression level of the gas vesicle gene gradually decreases. This indicates that the expression of gas vesicle gene (gvpC) and the formation of gas vesicle is a symbolic transition indicating that microcystis gets rid of a dormant state and regains buoyancy; after the algal cells are restored to normal cells, the continuous expression of the gas vesicle gene (gvpC) is not needed.

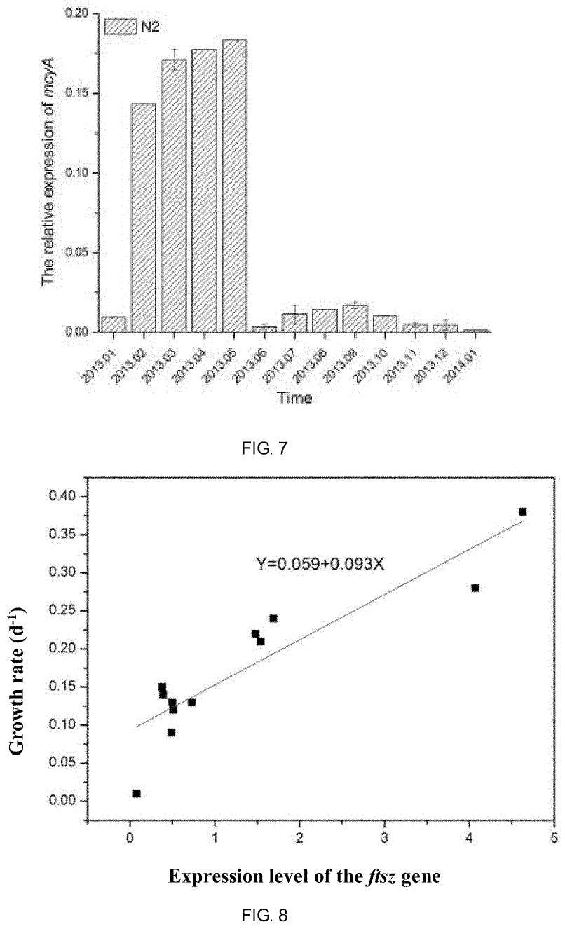

FIG. 7 shows the annual change in the relative expression level of the mcyA gene in the surface sediment of the Taihu Lake. The expression of the microcapsule algal toxin gene (mcyA) reaches its maximum value in February or March each year, and at particular points such as N4 and N5, the expression of mcyA has a significant correlation with the phycocyanin transcribed spacer gene and the gas vesicle gene and increases with the increase of the expressions of the phycocyanin transcribed spacer gene and gas vesicle gene.

Combined with FIG. 5, FIG. 6 and FIG. 7, the expression levels of three key genes including microcystis phycocyanin transcribed spacer gene (PC-IGS), gas vesicle synthesis gene (gvpC) and algal toxin synthesis gene (mcyA) in every lake area of the Taihu Lake begin to increase almost all in November, reach the maximum values in March and then show a downward trend from April. From the perspective of gene expression, the life history of cyanobacteria can be defined.

Step (3): Collect algae samples from complete water columns at different sampling points of the Taihu Lake, mix and filter them, quickly transfer the mixture to an RNase-free cryo-tube after concentration, put it in liquid nitrogen and bring it back to the laboratory for storage at −70° C. The sampling time is from March to June 2017. RNA is extracted from each sample and is subjected to reverse transcription to obtain microcystis cDNA.

RNA extraction step: Transfer a cryopreserved GF/C filter membrane to a lysing tube B (Lysing Matrix B, glass bead diameter 0.1 mm), add 500 μL of Buffer RLT, and place it in the Fast Prep-24 sample treatment instrument. Set the parameters of the Fast Prep-24 sample treatment instrument, 6 m/s, 35 s. After hitting and breaking, immediately take out the lysing tube and put it on ice, centrifuge it at 4° C., 12000 rpm for 10 s, and then transfer the supernatant to 2 mL of RNase-free centrifuge tube. Then operate according to the instructions of RNeasy Plant Mini Kit.

RNA reverse transcription: Use Transciptor First Strand cDNA Synthesis Kit reverse transcription kit for reverse transcription and calculate the volume needed for 400 ng according to the RNA concentration extracted from different samples. Perform reverse transcription according to the kit: Add 1 μL of Anchored-oligo(dT), 2 μL of random hexamer primers, 400 ng of RNA and an appropriate amount of PCR-grade water to make the total volume be 13 μL. After mixing, keep the mixture in a 65° C. warm bath of a PCR machine for 10 min. After the warm bathing, add 4 μL of transcriptor reverse transcriptase reaction buffer, 0.5 μL of RNA inhibitor, 2 μL of deoxynucleotide mix and 0.5 μL of transcriptor reverse transcriptase in turn, mix well and put the mixture on a PCR amplifier for reaction. Set the program of the PCR amplifier as 25° C. 10 min, 55° C. 30 min and 85° C. 5 min. Place the PCR tube on ice immediately after the reaction and store the obtained cDNA at −20° C. for later use.

Use the ftsZ gene and microcystis 16Sr RNA sequence primers to amplify the microcystis cDNA obtained at step (1);

After RNA extraction and reverse transcription of each sample, use the ftsZ and 16S rRNA primers in Table 1 to amplify the microcystis cDNA obtained through reverse transcription. The PCR reaction system is: 2 mmol/L dNTP mix 5 μL, 25 mmol/L Mg2+2 μL, 1.5 μL of upstream primers and 1.5 μL of downstream primers (10 μmol/L), 1 μL of high-fidelity DNA polymerase (TaKaRa, Japan) (1 U/μL), 5 μL of 10×PCR buffer, 3 μL of cDNA and deionized water up till 50 μL.

TABLE 1
Primers for amplifying microcystis ftsZ
and 16S rRNA genes

| Amplified | | |
|---|---|---|
| gene | Primer name | Primer sequence |
| ftsZ | ftsZF2 | GCTGAAGAATCGCGGGAAGA |
| | ftsZR4 | ATCCAGCATCGGCCATGAT |
| 16S rRNA | 184F | GCCGCRAGGTGAAAMCTAA |
| | 431R | AATCCAAARACCTTCCTCCC |

Amplify the ftsZ and 16SrRNA genes of each sample by fluorescent quantitative PCR, use 16Sr RNA as a housekeeping gene and ftsZ as a target gene, and calculate the relative expression level of the ftsZ gene in unit cells;

Establish a regression equation of microcystis growth rate μ and the relative expression level of the ftsZ gene through indoor culture experiment by using microcystis isolated from the algae samples collected from complete water columns, determine a rapid growth period of microcystis and obtain the relative expression level of the ftsZ gene at the moment as a threshold of the cyanobacteria rapid growth period window. If the relative expression level of the ftsZ gene is greater than this threshold, it is defined that cyanobacteria enter a lake cyanobacteria rapid growth period Separate the microcystis population taken from the Taihu Lake, use 4.0×10⁵ cell/L as the initial density and culture the microcystis in low temperature, low light, nutritive salt restriction, and suitable growth environment (Table 2) respectively, set three parallel controls for each treatment group, take samples every day and measure the density changes of algae cells by MUSE cell counter to obtain the growth rate of algae cells in different growth environments and in different growth cycles. The calculation formula of the growth rate is: ρ=(InNₜ−InN₀)/t, where Nₜ and N₀ represent the cell density at time t and the cell density at the initial time respectively).

The culture cycle is 30 days and the microcystis of each treatment group is sampled once every 3 days. The relative expression level of the ftsZ gene in unit cells is obtained according to the above method.

Finally, linear regression is performed on the growth rate μ of each sample and the relative expression level of corresponding ftsZ gene to obtain a regression equation of the growth rate of microcystis and the relative expression level of the ftsZ gene Y=0.059+0.093× (as shown in FIG. 8).

TABLE 2
Different growth environment conditions for indoor culture of
microcystis

| | Temperature | Luminous intensity (μmol | N, P concentration |
|---|---|---|---|
| No. | (° C.) | quanta m⁻² s⁻¹) | (mg/L) |
| 1 | 15 | 40 | N: 247, P: 5.4 |
| 2 | 25 | 10 | N: 247, P: 5.4 |
| 3 | 25 | 40 | N: 0.247, P: 0.054 |
| 4 | 25 | 40 | N: 247, P: 5.4 |

According to the regression equation of the growth rate μ of microcystis and the relative expression level of the ftsZ gene, the in situ growth rate of microcystis at the sampling points in different lake areas of the Taihu Lake is obtained.

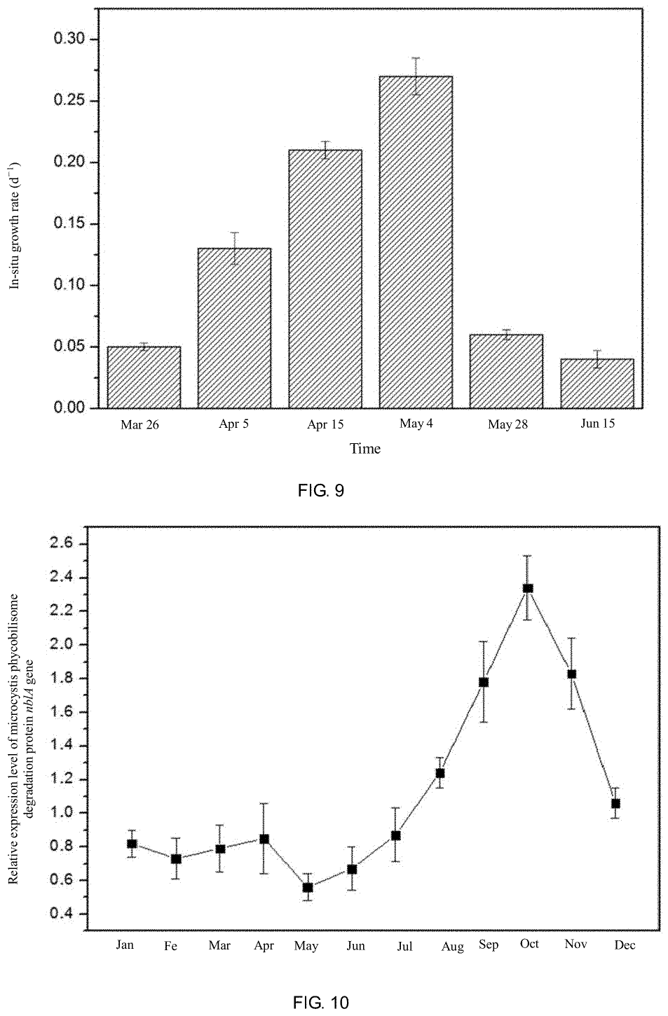

From the change law of in situ growth rate of microcystis from March to May in different lake areas of the Taihu Lake (FIG. 9), it can be seen that the growth rate of microcystis begins to increase from early April till May and then the growth begins to decline, so it is believed that the growth in the microcystis rapid growth period is about 0.13 d⁻¹. This value is used as a threshold for cyanobacteria rapid growth period, and the cyanobacteria rapid growth period is judged to be early April to the first ten days of May. According to FIG. 8, it can be obtained that the relative expression level of the ftsZ gene now is 0.5. This value is used as a threshold for cyanobacteria rapid growth period.

Step (4): The floating and aggregation of cyanobacteria is mainly affected by wind speed. The definition of cyanobacteria decline period in the present invention continues to use the research and discussion of the prior art on the vertical distribution of different algae in the water body under different meteorological and hydrological conditions (Kong Fanxiang et al. 2009). When the wind speed is greater than 3.1 m/s, cyanobacteria are mainly distributed in the water columns more than 0.5 m under the water surface, and when the wind speed is less than 3.1 m/s, most of the cyanobacteria will float on the water surface of the surface layer and form a cyanobacterial bloom, so wind speed 3. 1 m/s is defined as a threshold for bloom outbreak period.

Step (5): First, develop an in-situ sampling plan and make preparation for experiment; the microcystis used is Microcystis aeruginosa 7806, which is provided by the Freshwater Algae Culture Collection at the Institute of Hydrobiology (FACHB), China Center for Type Culture Collection of the Chinese Academy of Sciences.

Arrange sampling points in the lake, take in-situ samples from the lake month by month throughout the year and obtain complete water column samples at the sampling points;

From January 2016 to December 2016, collect complete water column algae samples at the Meiliang Bay in the northern part of the Taihu Lake every month, mix them on site immediately, filter 100 mL of water sample through a GF/C membrane, quickly transfer it to an RNase-free cryo-tube for storage, put the tube in liquid nitrogen, bring it back to the laboratory and store it at −70° C. until RNA extraction.

Perform RNA extraction and purification on the collected water column samples and the indoor purely cultured microcystis samples in a logarithmic phase and conduct RNA reverse transcription to obtain cDNA;

Indoor culturing of microcystis under optimal conditions:

Inoculate the microcystis aeruginosa species to a BG-11 medium and culture it in an illumination incubator. The culture conditions are: luminous intensity 40ρE·(m²·s)⁻¹, temperature 25° C., light to dark ratio 12 h: 12 h, shaking at fixed time 3 times a day;

Transfer the collected water column samples and the indoor purely cultured microcystis samples in a logarithmic phase to a 2 mL centrifuge tube that is RNase free and has been cooled in liquid nitrogen, and freeze and thaw the algae cells in liquid nitrogen repeatedly three times to break the cells.

Use RNeasy Plant Mini Kit of QIAGEN to extract RNA from algae cells. Extract RNA according to the instructions of the kit and determine RNA concentration.

Use a reverse transcription kit of QIAGEN to reversely transcribe RNA into cDNA.

Calculate the volume needed for 1 μg of RNA based on RNA concentration. Perform reverse transcription according to the kit: Add 2 μL of gDNA Wipeout Buffer, 1 μg of RNA and an appropriate amount of RNase-free H₂O, in a total volume of 14 μL. Turn the kit upside down, mix them well and put the mixture into a PCR amplifier and keep the mixture in a warm bath at 42° C. for 2 min to remove genomic DNA. Then add 1 μL of Reverse-transcription master mix, 4 μL of Quantiscript RT Buffer and 1 μL of RT Primer Mix, mix them, keep the mixture in a warm bath at 42° C. for 15 min in the PCR amplifier and then inactivate at 95° C. for 3 min.

Dilute the cDNA after reverse transcription 10 times as a fluorescent quantitative PCR template.

Determine the relative expression level of microcystis phycobilisome degradation protein nblA gene by the fluorescence quantitative PCR method;

Design amplification primers, as shown in Table 3:

TABLE 3
Primers for fluorescence quantitative PCR

| Gene name | Primer sequence | Function |
|---|---|---|
| 16S rRNA | F-5′- | Encode 16S |
| | GGACGGGTGAGTAACGCGTA-3′ | rRNA |
| | R-5′-CCCATTGCGGAAAATTCCCC-3′ | |
| nblA | F-5′- | Phycobilisome |
| | TTTTCTCTGACCATCATTTGTTCG-3′ | degradation |
| | R-5′- | |
| | CAGTTCAACATTCGTTCTTTTCAG-3′ | |

Fluorescence quantification uses the fluorescence quantification kit of QIAGEN. The reaction system is set to be 25 μL. The specific components in the system are: 1 μL of cDNA, 0.5 μL of forward primers, 0.5 μL of reverse primer, 12.5 μL of Reaction mix and 10.5 μL of RNase-free water. The reaction program is set to be: 95° C. 3 min, 95° C. 15 s/55° C. 45 s/72° C. 30 s (40 cycles). The gene expression level is evaluated with the Ct value of the reaction.

Amplify the microcystis phycobilisome degradation protein nblA gene by using the obtained water column sample cDNA as a fluorescent quantitative PCR template. After PCR, derive the corresponding threshold cycle value and record it as Ct_{field target gene}; amplify the 16srRNA internal reference gene of microcystis; after PCR, derive the corresponding threshold cycle value and record it as Ct_{same field sample 16s}; the difference between the two Ct_{field target gene}-Ct_{same field sample 16s} is the corrected cell copy number, recorded as ΔCt_{field target gene};

Amplify the microcystis phycobilisome degradation protein nblA gene by using the obtained indoor purely cultured microcystis sample cDNA as a fluorescent quantitative PCR template. After PCR, derive the corresponding threshold cycle value and record it as Ct_{indoor target gene}; amplify the 16srRNA internal reference gene of microcystis. After PCR, derive the corresponding threshold cycle value and record it as Ct_{same indoor sample 16s}; the difference between the two Ct_{indoor target gene}-Ct_{same indoor sample 16s} is the corrected cell copy number, which is recorded as ΔCt_{indoor target gent};

The relative expression level of the gene is represented with 2^{-ΔΔCt}, and the calculation formula of ΔΔCt is as follows:

ΔΔCt=ΔCt_{field target gene}−ΔCt_{indoor target gene}.

Determine the ratio of soluble glucose to neutral polysaccharides in the collected water column samples;

The collected water column samples are pre-filtered with a 0.45 μm filter element. Then the Pellicon tangential flow ultrafiltration system produced by Millipore, USA is used and the molecular weight intercepted by the ultrafiltration membrane is 1 kDa (equivalent to a pore size of 1 nm).

A total organic carbon analyzer (Shimadzu TOC-V CPN, Shimadzu) is used to determine the high molecular weight soluble organic carbon of the samples. The high molecular weight soluble organic powder obtained by freeze-drying is digested with 2M trifluoroacetic acid at 100° C. for 24 hours, and the newly prepared sodium borohydride (prepared with ammonia water) is added and the temperature is held at 40° C. for 1.5 hours so that the sugar (or sugar amine) is completely reduced into sugar alcohol and finally glacial acetic acid is added to stop the continued action of excess sodium borohydride. The mixed standard sugar sample is reduced by the same method. Take 0.4 mL of the sample, add 0.2 mL of methylimidazole and 1 mL of acetic anhydride, and acetylate at room temperature for 10 min. Add 5 mL of distilled water to degrade excess acetic anhydride. After cooling to room temperature with running tap water, add 1 mL of dichloromethane, mix them well, let stand for 10 min, suck away the aqueous phase in the upper layer, add 5 mL of distilled water, operate in the same way as above, discard the water layer and repeat the operation three times. After adding an appropriate amount of anhydrous sodium sulfate to the obtained dichloromethane phase in the lower layer, dehydrating and drying, centrifuge, blow dry dichloromethane by nitrogen blower, and replace it with n-hexane, which can be used to determine acetylated monosaccharide. Determination conditions of gas chromatography-mass spectrometer: Capillary column DB-225 (15 m×0.25 mm, 0.25 μm); column temperature: the initial temperature is 180° C. and the temperature is raised to 220° C. at 4° C., and held at 220° C. for 30 min. The injection volume is 2 μL, the carrier gas is argon and the flow rate is 2 mL/min. The standard samples of sugar are ribose, xylose, arabinose, rhamnose, fucose, galactose, glucose and mannose.

The calculated annual change diagram of the ratio of glucose to neutral polysaccharides in dissolved organic matter in the Taihu Lake is shown in FIG. 11.

Use time as the x-axis and the relative expression level of the nblA gene as the y-axis to draw a curve and determine a microcystis decline window period according to the curve of the relative expression level of the nblA gene over time.

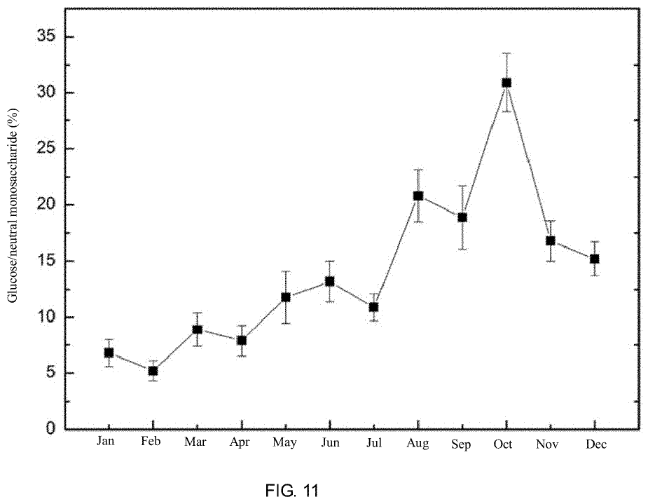

nblA is a gene encoding phycobilisome degradation protein. Phycobilisome degradation protein can degrade phycobilisome and affect the normal capture of light energy by algal cells. The annual change law of the relative ratio of nblA gene expression between wild cyanobacteria in the Taihu Lake and microcystis under the best indoor culture conditions shows that the ratio is low in winter and spring, about 0.8, but begins to increase in August at the end of summer, and reaches a peak in October, suggesting that the expression level of cyanobacteria phycobilisome degradation protein gene is the highest currently and the algae begin to decline rapidly. Then the relative expression level of the nblA gene begins to decrease (FIG. 10).

In comprehensive consideration of the relative expression level of the nblA gene and the ratio of glucose to neutral polysaccharides in dissolved organic matter, the relative expression level 2.4 of the nblA gene is determined as a threshold. When the relative expression level of the nblA gene reaches 2.4 and the ratio of dissolved glucose to neutral polysaccharides in the water body is greater than 30%, it is the first day of the cyanobacteria decline period.

What is claimed is:

1. A method for defining the stages of development of a cyanobacterial bloom, wherein the method comprises dividing the development of a cyanobacterial bloom into five stages: a cyanobacteria wintering period, a resuscitation period, a rapid growth period, an outbreak period and a cyanobacteria decline period; and the steps are as follows:
step (1): collect mixed water samples of water columns from the upper, middle and lower layers of the water body, overlying water samples and sediment samples at lake sampling points; determine the concentrations/contents of algocyan and chlorophyll a in the water columns, the overlying water and the sediment; calculate the concentration/content ratios of algocyan/chlorophyll a in the water columns, the overlying water and the sediment respectively; and define the time as a cyanobacteria wintering period when the concentration/content ratios of algocyan/chlorophyll a in the sediment, the overlying water and the water columns at each sampling point are all less than 1;
step (2): starting from the cyanobacteria wintering period, collect surface sediment samples from the lake sampling points, extract RNA from the surface sediment samples and determine the relative expression levels of phycocyanin synthesis gene PC-IGS, algal toxin synthesis gene mcyA and gas vesicle synthesis gene gvpC; and define that the cyanobacteria enter a resuscitation period when the relative expression levels of the genes satisfy relative expression level of PC-IGS>3, relative expression level of mcyA>0.03 and relative expression level of gvpC>0.004 at the same time;
step (3): starting from the cyanobacterial resuscitation period, collect complete water column algae samples from the lake sampling points, extract RNA and determine the relative expression level of the ftsZ gene; culture indoor the microcystis isolated from the same water column algae samples, establish a regression equation of microcystis growth rate μ and the relative expression level of the ftsZ gene, determine a rapid growth period of microcystis, obtain the relative expression level of the ftsZ gene at the moment as a threshold of the cyanobacteria rapid growth period window, and define that the cyanobacteria enter a lake cyanobacteria rapid growth period if the relative expression level of the ftsZ gene is greater than this threshold;
step (4): starting from the cyanobacteria rapid growth period, monitor the wind speed of each monitoring point in the lake, use 3.1 m/s as a threshold for defining an outbreak period and define the time as a cyanobacteria decline period when the wind speed is less than 3.1 m/s; and
step (5): starting from the cyanobacteria rapid growth period, collect complete water column samples from the lake sampling points, determine the relative expression level of the nblA gene and the ratio of glucose to neutral polysaccharides in dissolved organic matter, and define that the cyanobacteria enter a decline period when the relative expression level of the nblA gene reaches 2.4 and the ratio of dissolved glucose to neutral polysaccharides in the water body is greater than 30%.

2. The method for defining the stages of development of a cyanobacterial bloom according to claim 1, wherein at the step (1), the water sample from the upper layer of the water body refers to the water from the surface layer of the water body to 20 cm underwater; the water sample from the lower layer of the water body refers to the water 20 cm from the bottom layer; the middle section refers to a water sample from the middle layer of the water body; and the overlying water refers to the water in the surface layer of sediment.

3. The method for defining the stages of development of a cyanobacterial bloom according to claim 1, wherein the step (1) further comprises determining the sampling points in the lake as lake cyanobacteria wintering areas if the content ratio of algocyan/chlorophyll a in the sediment, the concentration ratio of algocyan/chlorophyll a in the overlying water and the concentration ratio of algocyan/chlorophyll a in the water columns at these points are all less than 1, and estimating and demarcating the area ranges based on the data of the monitoring points.

4. The method for defining the stages of development of a cyanobacterial bloom according to claim 1, wherein at the step (1), the earliest day when the concentration/content ratios of algocyan/chlorophyll a in the sediment, the overlying water and the water columns at each sampling point are all less than 1 is the first day of the cyanobacteria wintering period, and the 10 days before and after the first day of the cyanobacteria wintering period is a window period for taking control measures.

5. The method for defining the stages of development of a cyanobacterial bloom according to claim 1, wherein the step (2) further comprises sequencing according to the relative expression levels of the three genes at different sampling points of the lake, partitioning the lake through spatial grid division and spatial interpolation and determining the areas where the relative expression level of PC-IGS>3, the relative expression level of mcyA>0.03 and the relative expression level of gvpC>0.004 as resuscitation areas of wintering cyanobacteria.

6. The method for defining the stages of development of a cyanobacterial bloom according to claim 5, wherein GIS is used to layout grids, ArcGIS software is used for spatial interpolation and data storage and the inverse distance weight interpolation method is used for spatial interpolation to partition the lake.

7. The method for defining the stages of development of a cyanobacterial bloom according to claim 1, wherein at the step (2), starting from the cyanobacteria wintering period, the earliest day when the relative expression level of PC-IGS>3, the relative expression level of mcyA>0.03 and the relative expression level of gvpC>0.004 in surface sediment is the first day of the resuscitation period, and the 15 days before and after the first day of the resuscitation period is a window period for taking control measures.

8. The method for defining the stages of development of a cyanobacterial bloom according to claim 1, wherein at the step (3), the isolated microcystis population taken from the lake is divided into several treatment groups and cultured in different growth environments and samples are taken every day to measure the density changes of the algae cells to obtain growth rates of the algae cells in different growth environments and in different growth cycles; the calculation formula of the growth rate is: μ=(InNₜ−InN₀)/t, where Nₜ and N₀ represent the cell density at time t and the cell density at the initial time respectively; and the culture cycle is 30 days and microcystis in each treatment group is sampled to obtain the relative expression level of the ftsZ gene in unit cells.

9. The method for defining the stages of development of a cyanobacterial bloom according to claim 1, wherein at the step (5), neutral polysaccharides include ribose, xylose, arabinose, rhamnose, fucose, galactose, glucose and mannose.

10. The method for defining the stages of development of a cyanobacterial bloom according to claim 1, wherein the fluorescent quantitative PCR method is adopted to determine the relative expression levels of genes.
